(19) United States
(12) Patent Application Publication
(10) Pub. No.: US 2023/0040677 A1
(43) Pub. Date: Feb. 9, 2023

(54) SYSTEMS AND METHODS FOR AUTOMATED INSULIN DELIVERY RESPONSE TO MEAL ANNOUNCEMENTS

(71) Applicant: Tandem Diabetes Care, Inc., San Diego, CA (US)

(72) Inventors: Katherine Vyvy Tran, San Diego, CA (US); Jose Ricardo Rueda, La Jolla, CA (US); Nicholas Sherer, Escondido, CA (US); Thomas R. Ulrich, Oceanside, CA (US); Pat Mulvihill, San Diego, CA (US); Peter Zhao, San Diego, CA (US); Micah Stephens, San Diego, CA (US); Ryan Cardenas, San Diego, CA (US); Paul Harris, San Diego, CA (US); Geoffrey A. Kruse, San Diego, CA (US); Michael Michaud, San Diego, CA (US); Vance Swanson, San Diego, CA (US); Virginia S. Lu, San Diego, CA (US)

(21) Appl. No.: 17/879,959

(22) Filed: Aug. 3, 2022

Related U.S. Application Data
(60) Provisional application No. 63/228,891, filed on Aug. 3, 2021.

(51) Int. Cl. A61M 5/14

(52) U.S. Cl. CPC A61M 5/14; A61M 2005/14208

(57) ABSTRACT

An alternate mode for addressing meals in closed loop insulin delivery systems is disclosed. An Eating Soon Mode may be a user-selectable feature that can be activated when a user anticipates eating a meal in the near future. Once activated, Eating Soon Mode preemptively modifies the closed loop algorithm in preparation for the expected rise in glucose levels from consumption of the meal. This can result in a greater amount of time in range for the user, a lower coefficient of variation of the user's glucose levels and/or a lower maximum BG level for the user. According to embodiments disclosed herein, activation of Eating Soon Mode modifies the algorithm to deliver a correction bolus to lower the user's glucose level and to provide a modified, lower target range at which to maintain the user's glucose levels in anticipation of the glucose rise that will result from the meal.

### RELATED APPLICATION

The present application claims the benefit of U.S. Provisional Application No. 63/228,891, filed Aug. 3, 2021, which is hereby incorporated herein in its entirety by reference.

### FIELD OF THE INVENTION

The present invention relates generally to ambulatory infusion pumps and, more particularly, to operation of ambulatory infusion pumps in a closed-loop or semi-closed-loop fashion.

### BACKGROUND OF THE INVENTION

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as blood glucose meters (BGMs) and continuous glucose monitoring devices (CGMs). A CGM provides a substantially continuous estimated blood glucose level through a transcutaneous sensor that estimates blood analyte levels, such as blood glucose levels, via the patient's interstitial fluid CGM systems typically consist of a transcutaneously-placed sensor, a transmitter and a monitor.

Ambulatory infusion pumps typically allow the patient or caregiver to adjust the amount of insulin or other medicament delivered, by a basal rate or a bolus, based on blood glucose data obtained by a BGM or a CGM, and in some cases include the capability to automatically adjust such medicament delivery. Some ambulatory infusion pumps may include the capability to interface with a BGM or CGM such as, e.g., by receiving measured or estimated blood glucose levels and automatically adjusting or prompting the user to adjust the level of medicament being administered or planned for administration or, in cases of abnormally low blood glucose readings, reducing or automatically temporarily ceasing or prompting the user temporarily to cease or reduce insulin administration. These portable pumps may incorporate a BGM or CGM within the hardware of the pump or may communicate with a dedicated BGM or CGM via wired or wireless data communication protocols, directly and/or via a device such as a smartphone. One example of integration of infusion pumps with CGM devices is described in U.S. Patent Publication No. 2014/0276419, which is hereby incorporated by reference herein.

As noted above, insulin or other medicament dosing by basal rate and/or bolus techniques could automatically be provided by a pump based on readings received into the pump from a CGM device that is, e.g., external to the portable insulin pump or integrated with the pump as a pump-CGM system in a closed-loop or semi-closed-loop fashion. With respect to insulin delivery, some systems including this feature can be referred to as artificial pancreas systems because the systems serve to mimic biological functions of the pancreas for patients with diabetes. Such systems are also referred to as automated insulin delivery (AID) systems.

In some AID systems, a user can manually inform the algorithm that the user will be eating in what is often referred to as a “meal announcement.” In response to a meal announcement, some algorithms increase the basal rate being delivered to the user in anticipation of increased blood glucose levels due to the consumption of carbohydrates in the meal. Some closed loop systems alternatively or additionally enable a user to manually program a meal bolus by entering a number of carbohydrates estimated to be consumed or a number of units of insulin estimated to cover the anticipated amount of carbohydrates. However, increasing the basal rate in response to a meal announcement or delivering a meal bolus in response to a manual carbohydrate entry can subject the user to undesirable oscillations in glucose levels and/or hypoglycemia because the timing and amount of carbohydrates that will be consumed may be unknown.

### SUMMARY

Disclosed herein are apparatuses and methods that provide an alternate mode for addressing meals in closed loop insulin delivery systems. An Eating Soon Mode may be a user-selectable feature that can be activated when a user anticipates eating a meal in the near future. Once activated, Eating Soon Mode preemptively modifies the closed loop algorithm in preparation for the expected rise in glucose levels from consumption of the meal. This can result in a greater amount of time in range for the user, a lower coefficient of variation of the user's glucose levels and/or a lower maximum BG level for the user. According to embodiments disclosed herein, activation of Eating Soon Mode modifies the algorithm to deliver a correction bolus to lower the user's glucose level and to provide a modified, lower target range at which to maintain the user's glucose levels in anticipation of the glucose rise that will result from the meal.

In an embodiment, a system for closed loop diabetes therapy can include a pump mechanism configured to facilitate delivery of insulin to a user, a communications device adapted to receive glucose levels from a continuous glucose monitor, a user interface and at least one processor functionally linked to the pump mechanism, the user interface and the communications device. The at least one processor can be configured to automatically calculate insulin doses with a closed loop delivery algorithm based on glucose levels received from the continuous glucose monitor. The closed loop delivery algorithm can be configured to calculate the insulin doses to maintain the user's glucose levels within a default target glucose range including a low glucose threshold and a high glucose threshold and the calculated insulin doses can be automatically delivered to the user with the pump mechanism. User input selecting an eating soon mode configured to provide an indication that the user will be eating a meal in the near future from a device menu on the user interface can be received. In response to the selection of eating soon mode, a correction of bolus of insulin can be delivered to the user with the correction bolus of insulin configured to lower the user's glucose level to a predetermined eating soon target level below the low glucose threshold of the default target glucose range. The closed loop algorithm can also be modified to calculate and deliver insulin doses based on an eating soon target range having an eating soon low glucose threshold lower than the low glucose threshold of the default target range and an eating soon high glucose threshold lower than the high glucose threshold of the default target range. The eating soon mode can be terminated and the default target range used for the closed loop algorithm after a predetermined period of time or after receiving an indication through the user interface that the meal has been consumed.

In an embodiment, a system for closed loop diabetes therapy can include a pump mechanism configured to facilitate delivery of insulin to a user, a communications device adapted to receive glucose levels from a continuous glucose monitor, a user interface and at least one processor functionally linked to the pump mechanism, the user interface and the communications device. The at least one processor can be configured to automatically calculate insulin doses with a closed loop delivery algorithm based on glucose levels received from the continuous glucose monitor. The closed loop delivery algorithm can be configured to calculate the insulin doses to maintain the user's glucose levels within a default target glucose range including a low glucose threshold and a high glucose threshold and the calculated insulin doses can be automatically delivered to the user with the pump mechanism. Upon determining that a user will be consuming a meal in the near future the at least one processor can modify the default closed loop delivery algorithm in anticipation of an expected increase in glucose caused by the meal.

In an embodiment, a method of diabetes therapy can including automatically calculating insulin doses with a closed loop delivery algorithm based on glucose levels received from a continuous glucose monitor with the closed loop delivery algorithm configured to calculate the insulin doses to maintain the user's glucose levels within a default target glucose range including a low glucose threshold and a high glucose threshold. The calculated insulin doses can be automatically delivered to the user with a pump mechanism of an ambulatory infusion pump system. User input can be received selecting an eating soon mode from a device menu on a user interface of the ambulatory infusion pump system, the eating soon mode configured to provide an indication that the user will be eating a meal in the near future. A correction of bolus of insulin can be delivered to the user in response to the user input to lower the user's glucose level to a predetermined eating soon target level below the low glucose threshold of the default target glucose range. The closed loop algorithm can also be modified in response to the user input to calculate and deliver insulin doses based on an eating soon target range having an eating soon low glucose threshold lower than the low glucose threshold of the default target range and an eating soon high glucose threshold lower than the high glucose threshold of the default target range. Eating soon mode can be terminated and the default target range used for the closed loop algorithm after a predetermined period of time or after receiving an indication through the user interface that the meal has been consumed.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention may be more completely understood in consideration of the following detailed description of various embodiments of the invention in connection with the accompanying drawings, in which:

FIG. 1 is a medical device that can be used with embodiments of the disclosure.

FIG. 2 is a block diagram representing a medical device that can be used with embodiments of the disclosure.

FIGS. 3A-3B depict an embodiment of a pump system according to the disclosure.

FIG. 4 is a schematic representation of a system according to the disclosure.

FIG. 5 is a schematic representation of a closed-loop insulin delivery algorithm according to the disclosure.

FIG. 6 is a flowchart of a method of medicament delivery utilizing a closed loop delivery algorithm according to the disclosure.

FIG. 7 depicts a schematic representation of a method for closed loop insulin delivery according to an embodiment

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

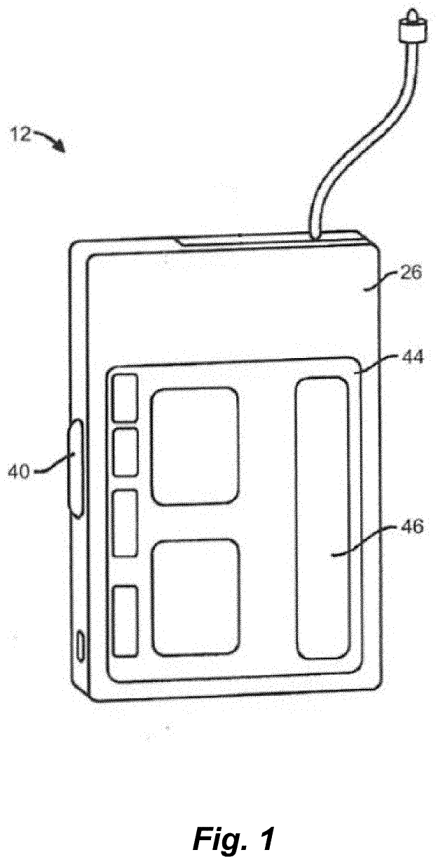

FIG. 1 depicts an embodiment of a medical device according to the disclosure. In this embodiment, the medical device is configured as a pump 12. Pump 12 may be an infusion pump that includes a pumping or delivery mechanism and reservoir for delivering medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display, a remote control device, a laptop computer, personal computer, tablet computer, a mobile communication device such as a smartphone, a wearable electronic watch or electronic health or fitness monitor, or personal digital assistant (PDA), a CGM display etc.

In an embodiment, the medical device can be an ambulatory insulin pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

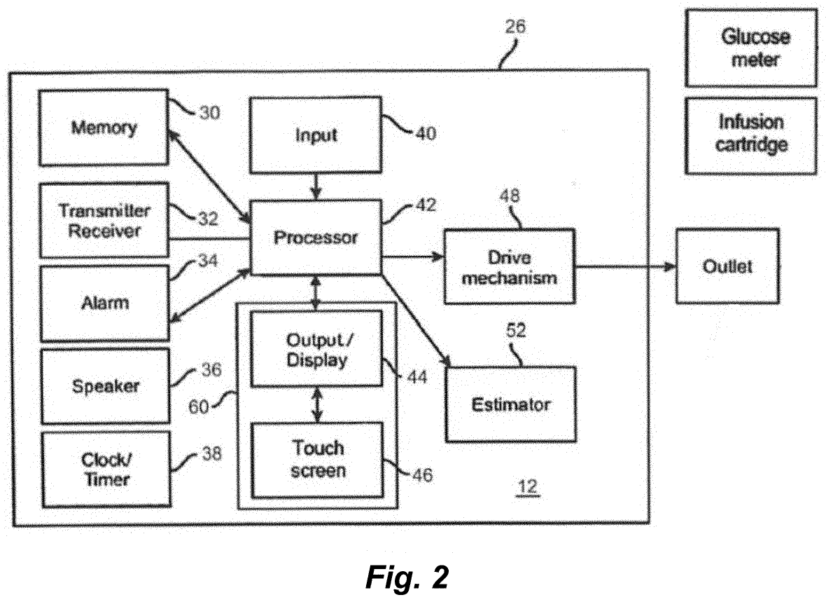

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor to receive signals and/or other data from an input device, such as a sensor that may sense pressure, temperature or other parameters.

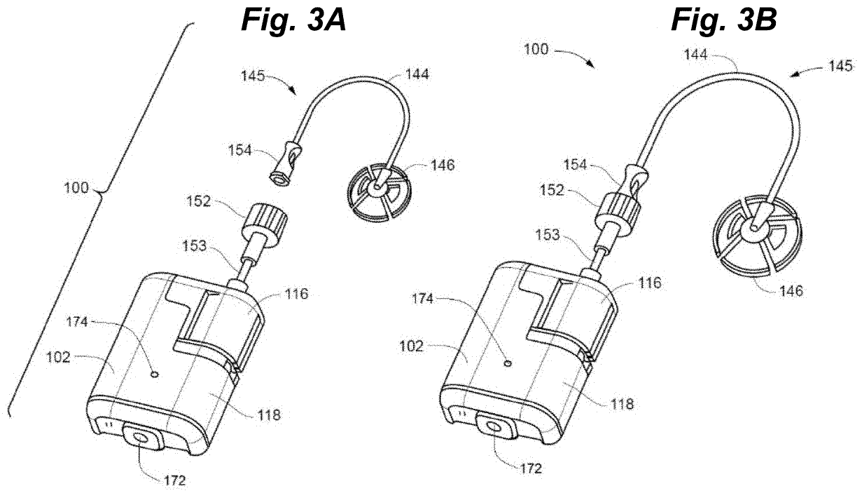

FIGS. 3A-3B depict another pump system including a pump 102 that can be used with embodiments. Drive unit 118 of pump 102 includes a drive mechanism 122 that mates with a recess in disposable cartridge 116 of pump 102 to attach the cartridge 116 to the drive unit 118. Pump system 100 can further include an infusion set 145 having a connector 154 that connects to a connector 152 attached to pump 102 with tubing 153. Tubing 144 extends to a site connector 146 that can attach or be pre-connected to a cannula and/or infusion needle that punctures the patient's skin at the infusion site to deliver medicament from the pump 102 to the patient via infusion set 145. In some embodiments, pump can include a user input button 172 and an indicator light 174 to provide feedback to the user.

In one embodiment, pump 102 includes a processor that controls operations of the pump and, in some embodiments, may receive commands from a separate device for control of operations of the pump. Such a separate device can include, for example, a dedicated remote control or a smartphone or other consumer electronic device executing an application configured to enable the device to transmit operating commands to the processor of pump 102. In some embodiments, processor can also transmit information to one or more separate devices, such as information pertaining to device parameters, alarms, reminders, pump status, etc. In one embodiment pump 102 does not include a display but may include one or more indicator lights 174 and/or one or more input buttons 172. Pump 102 can also incorporate any or all of the features described with respect to pump 12 in FIG. 2. Further details regarding such pumps can be found in U.S. Pat. No. 10,279,106 and U.S. Patent Publication Nos. 2016/0339172 and 2017/0049957, each of which is hereby incorporated herein by reference in its entirety.

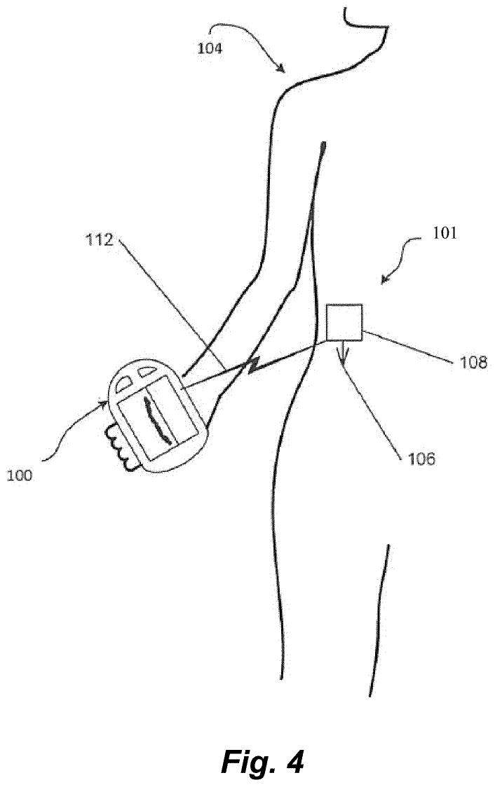

Pump 12 or 102 can interface directly or indirectly (via, e.g., a smartphone or other device) with a glucose meter, such as a blood glucose meter (BGM) or a continuous glucose monitor (CGM) or other glucose monitor. Referring to FIG. 4, an exemplary CGM system 100 according to an embodiment of the present invention is shown (other CGM systems can be used). The illustrated CGM system includes a sensor 101 affixed to a patient 104 that can be associated with the insulin infusion device in a CGM-pump system. The sensor 101 includes a sensor probe 106 configured to be inserted to a point below the dermal layer (skin) of the patient 104. The sensor probe 106 is therefore exposed to the patient's interstitial fluid or plasma beneath the skin and reacts with that interstitial fluid to produce a signal that can be associated with the patient's blood glucose (BG) level. The sensor 101 includes a sensor body 108 that transmits data associated with the interstitial fluid to which the sensor probe 106 is exposed. The data may be transmitted from the sensor 101 to the glucose monitoring system receiver 100 via a wireless transmitter, such as a near field communication (NFC) radio frequency (RF) transmitter or a transmitter operating according to a “Wi-Fi” or Bluetooth® protocol, Bluetooth® low energy protocol or the like, or the data may be transmitted via a wire connector from the sensor 101 to the monitoring system 100. Transmission of sensor data to the glucose monitoring system receiver by wireless or wired connection is represented in FIG. 4 by the arrow line 112. Further detail regarding such systems and definitions of related terms can be found in, e.g., U.S. Pat. Nos. 8,311,749, 7,711,402 and 7,497,827, each of which is hereby incorporated by reference in its entirety.

In an embodiment of a pump-CGM system having a pump 12, 102 that communicates with a CGM and that integrates CGM data and pump data as described herein, the CGM can automatically transmit the glucose data to the pump. The pump can then automatically determine therapy parameters and deliver medicament based on the data. Such an automatic pump-CGM system for insulin delivery can be referred to as an automated insulin delivery (AID) or an artificial pancreas system that provides closed-loop therapy to the patient to approximate or even mimic the natural functions of a healthy pancreas. In such a system, insulin doses are calculated based on the CGM readings (that may or may not be automatically transmitted to the pump) and are automatically delivered to the patient at least in part based on the CGM reading(s). In various embodiments, doses can be delivered as automated correction boluses and/or automated increases or decreases to a basal rate. Insulin doses can also be administered based on current glucose levels and/or predicted future glucoses levels based on current and past glucose levels.

For example, if the CGM indicates that the user has a high blood glucose level or hyperglycemia, the system can automatically calculate an insulin dose necessary to reduce the user's blood glucose level below a threshold level or to a target level and automatically deliver the dose. Alternatively, the system can automatically suggest a change in therapy upon receiving the CGM data such as an increased insulin basal rate or delivery of a bolus, but can require the user to accept the suggested change prior to delivery rather than automatically delivering the therapy adjustments.

If the CGM data indicates that the user has a low blood glucose level or hypoglycemia, the system can, for example, automatically reduce a basal rate, suggest to the user to reduce a basal rate, automatically deliver or suggest that the user initiate the delivery of an amount of a substance such as, e.g., a hormone (glucagon) to raise the concentration of glucose in the blood, automatically suggest that the user, e.g., ingest carbohydrates and/or take other actions and/or make other suggestions as may be appropriate to address the hypoglycemic condition, singly or in any desired combination or sequence. Such determination can be made by the infusion pump providing therapy or by a separate device that transmits therapy parameters to the infusion pump. In some embodiments, multiple medicaments can be employed in such a system as, for example, a first medicament, e.g., insulin, that lowers blood glucose levels and a second medicament, e.g., glucagon, that raises blood glucose levels.

As with other parameters related to therapy, such thresholds and target values can be stored in memory located in the pump or, if not located in the pump, stored in a separate location and accessible by the pump processor (e.g., “cloud” storage, a smartphone, a CGM, a dedicated controller, a computer, etc., any of which is accessible via a network connection). The pump processor can periodically and/or continually execute instructions for a checking function that accesses these data in memory, compares them with data received from the CGM and acts accordingly to adjust therapy. In further embodiments, rather than the pump determining the therapy parameters, the parameters can be determined by a separate device and transmitted to the pump for execution. In such embodiments, a separate device such as the CGM or a device in communication with the CGM, such as, for example, a smartphone, dedicated controller, electronic tablet, computer, etc. can include a processor programmed to calculate therapy parameters based on the CGM data that then instruct the pump to provide therapy according to the calculated parameters.

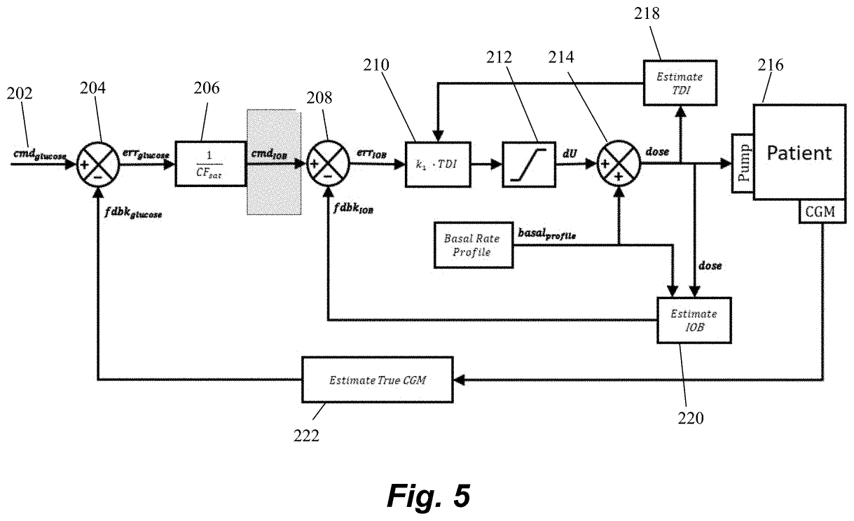

A schematic representation of a control algorithm for automatically adjusting insulin delivery based on CGM data is depicted in FIG. 5. This figure depicts an algorithm for increasing basal rate that utilizes a cascaded loop. The logic for decreasing basal rate is not depicted. In the depicted embodiment, there is a glucose set-point/command (cmd) that is determined at step 202. The glucose set point is a target value at which the algorithm attempts to maintain a user's blood glucose. This value can vary based on a number of factors, including the user's physiology, whether the user is awake or asleep, how long the user has been awake, etc. The glucose set point is compared to the actual CGM feedback (fdbk) at step 204 to determine a glucose error value (err) that is the difference between the set point and the feedback. The errGLUCOSE value at step 206 is multiplied by a constant (1/CF), in which CF is the user's correction factor, or amount by which one unit of insulin lowers the user's blood glucose. This calculation determines how much insulin is needed to correct the glucose error, which is how much insulin on board (IOB) is needed in the user's body. This IOB value then determines an appropriate estimated insulin on board (IOB) set point for the patient.

The estimated IOB level determined at step 206 is then taken as the command (cmdIOB) for the inner loop and based on a difference of an IOB feedback value (fdbkIOB) and the cmdIOB set point at step 208, an IOB error value (errIOB) is determined. At step 210, the errIOB value is multiplied by a constant k1 (relating to insulin-dependent glucose uptake in the body) and an estimate of the total daily insulin (TDI) of the user. This adjusts the errIOB to be proportional to the constant and the user's total daily intake of insulin. At step 212, a limiter function is applied to the value calculated at step 210. The limiter function prevents the calculated amount from being larger or smaller than preset limits. The result is an insulin amount dU, which is the amount by which the user's stored basal rate should be modified. The insulin delivery rate for the user for the next closed loop interval is therefore calculated by modifying the user's stored basal rate profile by the dU value at step 214.

After the dose is calculated, it can be delivered to the user at step 216 and can also be used to update the estimated TDI for the user at step 218. The dose can also be used to update the estimated IOB level for the user at step 220 by comparing the actual insulin delivered to the programmed basal rate. The updated estimated IOB then becomes the new fdbkIOB for the IOB comparison at step 208. When new CGM values are received from the CGM, an estimated true CGM can be determined based on various factors such as, for example, the calibration status of the CGM sensor, and the estimated true CGM value then becomes the new fdbkGLUCOSE value for the outer loop comparison with cmdGLUCOSE at step 204. The algorithm then proceeds through to calculate a new estimated IOB and to the inner IOB loop for calculation of an insulin dose as described above. In an embodiment, a new CGM value is received every 5 minutes and therefore the algorithm executes as set forth above every 5 minutes.

While closed loop algorithms such as the algorithm described with respect to FIG. 5 can vary insulin delivery according to measured glucose levels, these systems typically require additional measures for addressing the significant rise in glucose levels caused by consumption of meals, such as manual programming of meal boluses and/or meal announcements that cause the system to increase the level of basal insulin. However, as noted above, these approaches can lead to undesirable oscillations in glucose levels. Systems and methods disclosed herein therefore provide an alternative Eating Soon Mode for the closed loop algorithm that modifies the algorithm when activated by a user to preemptively address the planned meal to increase the time spent in euglycemia following consumption of the meal.

The Eating Soon Mode may be a user-selectable feature that can be activated by a user when a user anticipates eating a meal in the near future. For example, the Eating Soon Mode can be configured to be activated when the user anticipates eating within, e.g., an hour, 30 minutes, etc. The Eating Soon Mode can be activated by selection of a menu item activating the mode on a programming menu displayed on, e.g., the user's pump, a remote control device for controlling the pump, a smartphone or other device executing a software application for control of the pump, etc. Once activated, Eating Soon Mode preemptively modifies the closed loop algorithm in preparation for the expected rise in glucose levels from consumption of the meal. This can result in a greater amount of time in range for the user, a lower coefficient of variation of the user's glucose levels and/or a lower maximum BG level for the user.

As noted above, closed loop algorithms attempt to maintain the user's current or predicted future glucose levels at a glucose target or within a glucose range, such as, for example, between 112.5 mg/dL and 160 mg/dL. According to embodiments disclosed herein, activation of Eating Soon Mode causes the system to modify the algorithm to deliver a correction bolus to lower the user's glucose level and to provide a modified, lower range at which to maintain the user's glucose levels in anticipation of the glucose rise that will result from the meal. For example, upon activation of Eating Soon Mode the algorithm can automatically cause a correction bolus to be delivered that will lower the user's glucose level to an Eating Soon Target of, for example, 100 mg/dL, that may be below the typical range between which the algorithm generally attempts to maintain the user's glucose levels. Eating Soon Mode can then also modify the target range to a lower and/or narrower range, such as, for example, 80 mg/dL to 100 mg/dL. In embodiments, to maintain the user's glucose level at the lower range, the basal rate delivered to the user can initially be increased from the default rate, such as, for example, to 1.4 times the default rate and then continually adjusted based on glucose feedback. Eating Soon Mode is therefore configured to maintain the user's glucose levels at a lower level, e.g., as low as is safely possible without causing hypoglycemia, to prepare for the anticipated increase in glucose that will be caused by the meal. When the user programs a meal bolus or otherwise provides an indication to the system that the meal is going to be or was consumed, the pump can automatically exit Eating Soon Mode and return to the default algorithm settings and target glucose range. In some embodiments, the meal bolus is delivered as programmed (i.e., units delivered=carbohydrates consumed/carbohydrate ratio) without subtracting from or otherwise modifying the bolus for the extra insulin delivered by Eating Soon Mode. In other embodiments, the meal bolus can be reduced by the extra insulin delivered to the user from Eating Soon Mode and/or the user's insulin on board. A user may also be able to manually instruct the system to exit Eating Soon Mode.

If the user does not program a meal bolus within a predetermined meal period for Eating Soon Mode, such as, for example, 30 minutes, 1 hour, etc., the system can automatically terminate Eating Soon Mode. In some embodiments, in response to the termination of Eating Soon Mode due to the user not indicating that the meal was consumed, the system will enter a Safeguard Mode that increases the target glucose range for a predetermined period of time to compensate for the extra insulin delivered during Eating Soon Mode that was not needed because the meal was not consumed. For example, Safeguard Mode can apply a higher and narrower range than the default range for the algorithm such as, for example, between 140 mg/dL and 160 mg/dL. Safeguard Mode can then be automatically deactivated and the algorithm can return to the default glucose level range after a predetermined period of time. In some embodiments, Safeguard Mode can maintain the elevated glucose range for a shorter period of time than Eating Soon Mode maintains the lowered glucose range, such as, for example, Safeguard Mode being effective for 40 minutes following a 1 hour Eating Soon Mode, Safeguard Mode for 20 minutes following a 30 minute Eating Soon Mode, etc.

In various embodiments, the glucose ranges and/or time periods for Eating Soon Mode and/or Safeguard can be preset on the device or can be user-selectable. For example, a user may be able to select low and high glucose levels for the Eating Soon Mode target range of between about 50 and 160 mg/dL for the low target and about 100 and 200 mg/dL for the high target and a time period of 15 minutes, 30 minutes or 1 hour, for example. Similarly, for Safeguard Mode a user may be able to select low and high glucose levels for the target range of between about 100 and 160 mg/dL for the low target and about 120 and 200 mg/dL for the high target and a time period of 10 minutes, 20 minutes, or 40 minutes, for example. Alternatively, the glucose level range and/or time period for Safeguard Mode can automatically be determined based on the settings for Eating Soon Mode, such as, for example, the time period being a predetermined percentage of the time period for Eating Soon Mode. A user may also be able to select the glucose target to which the initial correction bolus of Eating Soon Mode is intended to lower the user's glucose level. In some embodiments, the correction bolus target glucose level and the upper limit of the glucose target range for Eating Soon Mode are automatically equal to each other.

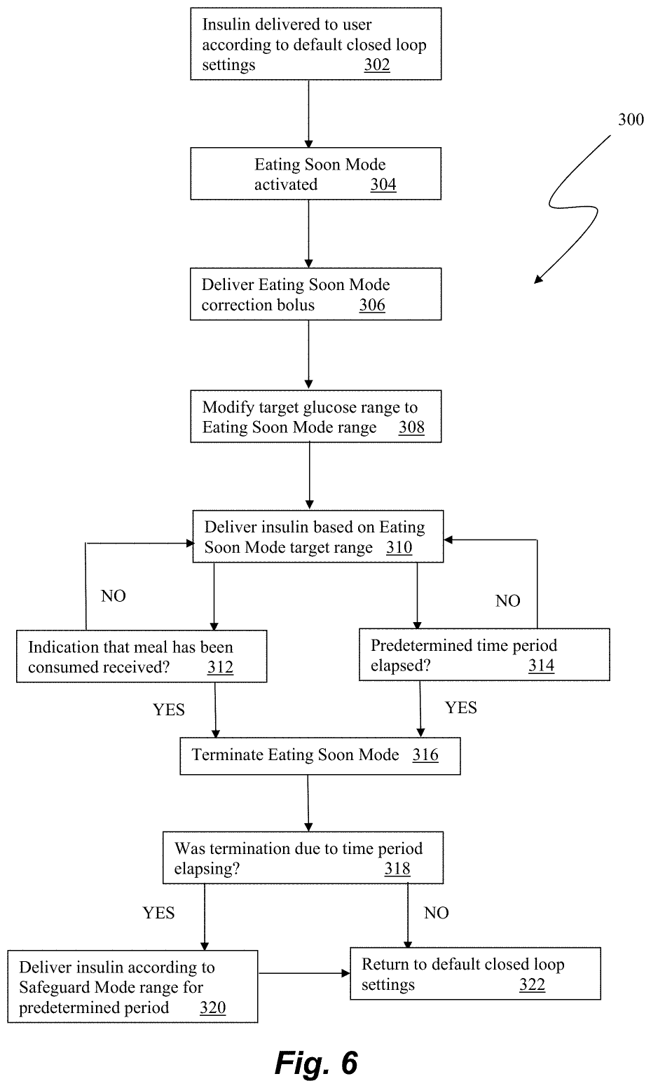

Referring now to FIG. 6, a flowchart of a method of medicament delivery 300 utilizing a closed loop delivery algorithm according to the disclosure is depicted. At step 302, the closed loop delivery algorithm is calculating insulin doses that are delivered to the user based on glucose levels according to its standard programmed and/or default settings. The user activates Eating Soon Mode at step 304. Upon activation of Eating Soon Mode, the algorithm can deliver a correction bolus to the predetermined Eating Soon Target level at step 306 and can modify the glucose target range to the lower Eating Soon Mode target range at step 308. For example, the algorithm can cause a correction bolus to be delivered to lower the user's glucose level to 100 mg/dL and then operate the system to maintain the user's glucose level between 80 mg/dL and 100 mg/dL. Insulin is then delivered to the user according to the Eating Soon Mode target range at step 310.

The system continuously determines whether an indication that the meal has been consumed has been received at step 312 and whether the predetermined time period for Eating Soon Mode has elapsed at step 314. If no indication that the meal has been consumed has been received and the predetermined time period has not elapsed, the system continues delivering insulin in the Eating Soon Mode at step 310. If an indication that the meal has been consumed has been received, such as by the user programming a meal bolus, or if the predetermined time period has elapsed, Eating Soon Mode is terminated at step 316. The system then determines at step 318 if Eating Soon Mode was terminated due to the predetermined time period elapsing. If not (i.e. Eating Soon Mode was terminated because the user indicated the meal was being consumed), the system returns to the default closed loop settings at step 322. If the termination was due to the predetermined time period elapsing, the system activates Safeguard Mode and calculates insulin delivery based on the Safeguard Mode range for a predetermined period of time at step 320. Once the predetermined period of time for Safeguard Mode has elapsed, the system returns to the default delivery settings at step 322.

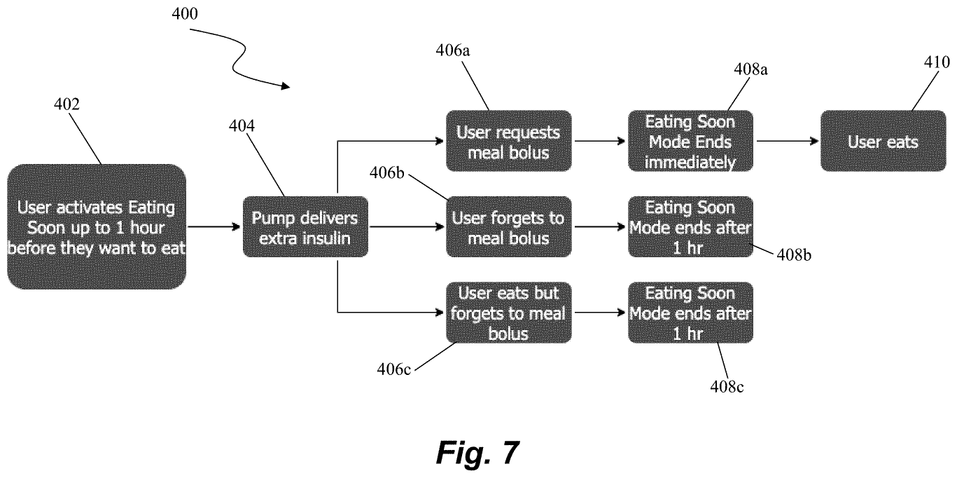

FIG. 7 depicts a schematic representation of a method for closed loop insulin delivery 400 according to an embodiment. When a user activates Eating Soon Mode at step 402, the system delivers extra insulin to the user, such as, for example, as described herein by delivering an initial correction bolus and maintaining a lower glucose level range, at step 404. The system continues to deliver the extra insulin until the user either requests a meal bolus 406a, doesn't eat and therefore doesn't request a meal bolus 406b, or eats but forgets to request a meal bolus 406c. If the user does not request a bolus, either because the user didn't eat 406b or because the user ate and forgot to request a bolus 406c, the system automatically terminates Eating Soon Mode after one hour 408b, 408c. If the user does program a meal bolus 406a, Eating Soon Mode is ended immediately 408a as the user then proceeds to eat 410. In practice, Eating Soon Mode can be particularly useful when, for example, a user is in a restaurant after ordering while waiting for food to come out, for children while a parent is preparing food, or any other time when it is known that the user will be eating soon.

In embodiments, a system for closed loop diabetes therapy can include a pump mechanism configured to facilitate delivery of insulin to a user, a communications interface adapted to receive glucose levels from a continuous glucose monitor, a user interface and at least one processor functionally linked to the pump mechanism, the user interface and the communications interface. The at least one processor can be configured to automatically calculate insulin doses with a closed loop delivery algorithm based on glucose levels received from the continuous glucose monitor to maintain the user's glucose levels within a default target glucose range including a low glucose threshold and a high glucose threshold and to cause the pump mechanism to automatically deliver the calculated insulin doses to the user. If the processor receives an indication that the user will be eating a meal in the near future, a correction bolus of insulin configured to lower the user's glucose level to a predetermined target level can be delivered and the closed loop algorithm can be modified to calculate and deliver insulin doses based on a modified target range having a modified low glucose threshold lower than the low glucose threshold of the default target range and a modified high glucose threshold lower than the high glucose threshold of the default target range.

In some embodiments, the at least one processor is further configured to resume delivering insulin according to the default target range for the closed loop algorithm automatically after a predetermined period of time following the indication that the user will be eating a meal in the near future.

In some embodiments, the processor is further configured to calculate and deliver insulin doses according to a safeguard target range having one or more thresholds that are higher than the default target range after the predetermined period of time prior to resuming delivering insulin according to the default target range.

In some embodiments, the at least one processor is further configured to resume delivering insulin according to the default target range for the closed loop algorithm upon determining that the meal has been consumed.

In some embodiments, the at least one processor is configured to determine that the meal has been consumed when a meal bolus has been programmed.

In some embodiments, the programmed meal bolus is reduced based on one or more of an amount of the correction bolus delivered in response to the indication and an amount of increased insulin delivered according to the modified target range.

In some embodiments, the at least one processor is configured to deliver the meal bolus as programmed.

In some embodiments, the at least one processor is configured to receive an indication that the user will be eating a meal in the near future via user input selecting an eating soon mode from a device menu on the user interface.

In some embodiments, the predetermined target level is lower than the low glucose threshold of the default target range.

In embodiments, a system for closed loop diabetes therapy can include a pump mechanism configured to facilitate delivery of insulin to a user, a communications interface adapted to receive glucose levels from a continuous glucose monitor, a user interface and at least one processor functionally linked to the pump mechanism, the user interface and the communications interface. The at least one processor can be configured to automatically calculate insulin doses with a default closed loop delivery algorithm based on glucose levels received from the continuous glucose monitor to maintain the user's glucose levels within a default target glucose range including a low glucose threshold and a high glucose threshold and to cause the pump mechanism to automatically deliver the calculated insulin doses to the user. If the at least one processor receives an indication that the user will be eating a meal in the near future, the default closed loop delivery can be modified in anticipation of an expected increase in glucose caused by the meal.

In some embodiments, wherein the processor is configured to modify the closed loop delivery algorithm in response to the indication that the user will be eating the meal in the near future in anticipation of an expected increase in glucose caused by calculating and delivering insulin doses based on a modified target range having a modified low glucose threshold lower than the low glucose threshold of the default target range and a modified high glucose threshold lower than the high glucose threshold of the default target range.

In some embodiments, the processor is further configured to deliver a correction bolus configured to lower the user's glucose level to a predetermined target level in response to the indication that the user will be eating the meal in the near future.

In some embodiments, the predetermined target level is lower than the low glucose threshold of the default target range.

In some embodiments, the at least one processor is further configured to resume delivering insulin according to the default closed loop delivery algorithm automatically after a predetermined period of time following the indication that the user will be eating a meal in the near future.

In some embodiments, the processor is further configured to calculate and deliver insulin doses according to a safeguard target range having one or more thresholds that are higher than the default target range after the predetermined period of time prior to resuming delivering insulin according to the default target range.

In some embodiments, the at least one processor is further configured to resume delivering insulin according to the default close loop delivery algorithm upon determining that the meal has been consumed.

In some embodiments, the at least one processor is configured to determine that the meal has been consumed when a meal bolus has been programmed.

In some embodiments, the programmed meal bolus is reduced based on increased insulin delivered based on modify the default closed loop delivery algorithm.

In some embodiments, the at least one processor is configured to deliver the meal bolus as programmed.

In some embodiments, the at least one processor is configured to receive an indication that the user will be eating a meal in the near future via user input selecting an eating soon mode from a device menu on the user interface.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; and 11,305,057 and commonly owned U.S. Patent Publication Nos. 2009/0287180; 2012/0123230; 2013/0053816; 2014/0276423; 2014/0276569; 2014/0276570; 2018/0071454; 2019/0240398; 2019/0307952; 2020/0206420; 2020/0261649; 2020/0329433; 2020/0368430; 2020/0372995; 2021/0001044; 2021/0113766; 2021/0154405; 2021/0353857; 2022/0062553; 2022/0139522 and 2022/0223250 and commonly owned U.S. patent applications Ser. Nos. 17/368,968; 17/587,412; 17/587,434; 17/587,468; 17/677,621; 17/729,464; and 17/732,208.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. A system for closed loop diabetes therapy, comprising:
a pump mechanism configured to facilitate delivery of insulin to a user;
a communications interface adapted to receive glucose levels from a continuous glucose monitor;
a user interface; and
at least one processor functionally linked to the pump mechanism, the user interface and the communications interface, the at least one processor configured to:
automatically calculate insulin doses with a closed loop delivery algorithm based on glucose levels received from the continuous glucose monitor, the closed loop delivery algorithm configured to calculate the insulin doses to maintain the user's glucose levels within a default target glucose range including a low glucose threshold and a high glucose threshold;
cause the pump mechanism to automatically deliver the calculated insulin doses to the user;
receive an indication that the user will be eating a meal in the near future;
deliver a correction bolus of insulin in response to the indication, the correction bolus of insulin configured to lower the user's glucose level to a predetermined target level; and
modify the closed loop algorithm in response to the indication to calculate and deliver insulin doses based on a modified target range having a modified low glucose threshold lower than the low glucose threshold of the default target range and a modified high glucose threshold lower than the high glucose threshold of the default target range.

2. The system of claim 1, wherein the at least one processor is further configured to resume delivering insulin according to the default target range for the closed loop algorithm automatically after a predetermined period of time following the indication that the user will be eating a meal in the near future.

3. The system of claim 2, where the processor is further configured to calculate and deliver insulin doses according to a safeguard target range having one or more thresholds that are higher than the default target range after the predetermined period of time prior to resuming delivering insulin according to the default target range.

4. The system of claim 1, wherein the at least one processor is further configured to resume delivering insulin according to the default target range for the closed loop algorithm upon determining that the meal has been consumed.

5. The system of claim 4, wherein the at least one processor is configured to determine that the meal has been consumed when a meal bolus has been programmed.

6. The system of claim 5, wherein the programmed meal bolus is reduced based on one or more of an amount of the correction bolus delivered in response to the indication and an amount of increased insulin delivered according to the modified target range.

7. The system of claim 5, wherein the at least one processor is configured to deliver the meal bolus as programmed.

8. The system of claim 1, wherein the at least one processor is configured to receive an indication that the user will be eating a meal in the near future via user input selecting an eating soon mode from a device menu on the user interface.

9. The system of claim 1, wherein the predetermined target level is lower than the low glucose threshold of the default target range.

10. A system for closed loop diabetes therapy, comprising:
a pump mechanism configured to facilitate delivery of insulin to a user;
a communications interface adapted to receive glucose levels from a continuous glucose monitor;
a user interface; and
at least one processor functionally linked to the pump mechanism, the user interface and the communications interface, the at least one processor configured to:
automatically calculate insulin doses with a default closed loop delivery algorithm based on glucose levels received from the continuous glucose monitor, the default closed loop delivery algorithm configured to calculate the insulin doses to maintain the user's glucose levels within a default target glucose range including a low glucose threshold and a high glucose threshold;
cause the pump mechanism to automatically deliver the calculated insulin doses to the user;
receive an indication that the user will be eating a meal in the near future; and
modify the default closed loop delivery algorithm in response to the indication that the user will be eating the meal in the near future in anticipation of an expected increase in glucose caused by the meal.

11. The system of claim 10, wherein the processor is configured to modify the closed loop delivery algorithm in response to the indication that the user will be eating the meal in the near future in anticipation of an expected increase in glucose caused by calculating and delivering insulin doses based on a modified target range having a modified low glucose threshold lower than the low glucose threshold of the default target range and a modified high glucose threshold lower than the high glucose threshold of the default target range.

12. The system of claim 10, wherein the processor is further configured to deliver a correction bolus configured to lower the user's glucose level to a predetermined target level in response to the indication that the user will be eating the meal in the near future.

13. The system of claim 12, wherein the predetermined target level is lower than the low glucose threshold of the default target range.

14. The system of claim 10, wherein the at least one processor is further configured to resume delivering insulin according to the default closed loop delivery algorithm automatically after a predetermined period of time following the indication that the user will be eating a meal in the near future.

15. The system of claim 14, where the processor is further configured to calculate and deliver insulin doses according to a safeguard target range having one or more thresholds that are higher than the default target range after the predetermined period of time prior to resuming delivering insulin according to the default target range.

16. The system of claim 10, wherein the at least one processor is further configured to resume delivering insulin according to the default close loop delivery algorithm upon determining that the meal has been consumed.

17. The system of claim 16, wherein the at least one processor is configured to determine that the meal has been consumed when a meal bolus has been programmed.

18. The system of claim 17, wherein the programmed meal bolus is reduced based on increased insulin delivered based on modify the default closed loop delivery algorithm.

19. The system of claim 17, wherein the at least one processor is configured to deliver the meal bolus as programmed.

20. The system of claim 10, wherein the at least one processor is configured to receive an indication that the user will be eating a meal in the near future via user input selecting an eating soon mode from a device menu on the user interface.
